(19) United States
(12) Patent Application Publication
(10) Pub. No.: US 2024/0274270 A1
(43) Pub. Date: Aug. 15, 2024

(54) COMMUNICATION INTERFACE ADAPTED ACCORDING TO A COGNITIVE EVALUATION FOR SPEECH-IMPAIRED PATIENTS

(71) Applicant: CENTRE HOSPITALIER REGIONAL UNIVERSITAIRE DE TOURS, Tours (FR)

(72) Inventors: Laetitia BODET-CONTENTIN, LA RICHE (FR); Stephan EHRMANN, TOURS (FR)

(21) Appl. No.: 18/568,634

(22) PCT Filed: Jun. 13, 2022
(86) PCT No.: PCT/FR2022/051120

§ 371 Date: Dec. 8, 2023

(30) Foreign Application Priority Data
Jun. 14, 2021 (FR) FR2106239

(51) Int. Cl. G16H 20/70; G06F 3/01; G06F 3/04817

(52) U.S. Cl. CPC G16H 20/70; G06F 3/013; G06F 3/04817

(57) ABSTRACT

The invention relates to a communication method, comprising the steps of: providing a patient with a control device and a display screen; executing a sequence of cognitive tests to determine a comprehension score (SCR) of the patient; selecting a communication interface as a function of the comprehension score, from a plurality of communication interfaces (EINT, SINT, CINT) having distinct respective complexities, each communication interface comprising at least one home page with selectable areas of interest using the control device; activating the selected communication interface and displaying the home page of the selected communication interface on the display screen; detecting the designation by the control device of an area of interest among the areas of interest in a page displayed on the display screen; and emitting a voice message following the detection of the designation of an area of interest.

The present invention relates to a communication interface device suitable in particular for patients hospitalized in critical care departments (intensive care, continuous monitoring).

In fact, the majority of patients in intensive care units are deprived of speech due to the presence of an intubation tube between their vocal cords, or a tracheotomy without a phonation cannula. However, 50% are conscious and therefore able to communicate. In addition, some patients suffer from resuscitation tetraparesis, making it extremely difficult for them to mobilize themselves sufficiently to use non-verbal means of communication such as writing or using a computer keyboard. Lying down also makes it difficult to use a keyboard.

Communication difficulties can also result from neurological disorders such as confusional or dementing syndromes, resuscitation delirium, neurological lesions, or the pharmacological or secondary effects of drugs.

For patients, this inability to communicate while conscious is a source of frustration, dehumanization, depersonalization, loss of self-esteem and stress. As a result, resuscitation can be experienced as torture, leading to post-traumatic stress disorder and significant short- and long-term psychological sequelae.

This difficulty in communicating also leaves caregivers unable to understand their patients' needs and demands. For the patients' relatives, it also leads to a feeling of powerlessness.

There is therefore a need to enable patients who have lost the ability to speak and use their hands to communicate with caregivers and relatives.

It has been proposed to use an optical eye-tracking device to control a computer. For example, patent application US 2019/0272718 describes a device intended for use by a patient who is bedbound, intubated, and unable to move. This device comprises a support configured to hold a display screen in front of the patient's face, an eye-tracking device, and a speech synthesis device.

However, this device can be tedious to use on a patient who is particularly weak and rapidly exhausted. What's more, a patient's condition in the intensive care unit can improve or deteriorate rapidly. Generally speaking, a patient's ability to communicate can vary greatly from one moment to the next in his or her care.

In this context, it is advisable to offer patients a means of communication that is effective and adapted to their cognitive abilities and ability to concentrate during their care.

Embodiments relate to a communication method, comprising the following steps carried out by a computer: providing a patient with a control device and a display screen connected to the computer; executing a sequence of cognitive tests during which the patient interacts with the computer using the display screen and the control device, to determine a comprehension score for the patient; selecting a communication interface as a function of the comprehension score, from a plurality of communication interfaces implemented by the computer and having distinct respective complexities, each communication interface including at least one home page displayable on the display screen and including selectable areas of interest using the control device; activating the selected communication interface and displaying the home page of the selected communication interface; detecting a designation by the control device of an area of interest among the areas of interest in a page displayed on the display screen; and controlling the emission of a voice message following detection of the designation of an area of interest.

According to an embodiment, the communication interfaces comprise: a first communication interface comprising only a home page providing access to at most ten commands, and/or a second communication interface providing access to commands from areas of interest distributed over the home page and several other pages, the commands of the second communication interface being available in at most six selections using the control device, and/or a third communication interface providing access to commands distributed over an unlimited number of pages.

According to an embodiment, each of the cognitive tests comprises steps of: displaying a page comprising several images on the display screen; issuing a voice command in relation to the images on the displayed page; acquiring a response from the patient in relation to the page displayed, using the control device; and updating the comprehension score as a function of the patient's response, wherein the patient's response is a selection signal for an area of the display screen or eye movements detected using an eye-tracking device.

According to an embodiment, the method comprises the incrementation by the computer of the score when the patient has maintained his/her gaze on one of the images displayed, corresponding to the vocal instruction.

According to an embodiment, the cognitive tests comprise word comprehension tests, simple sentence comprehension tests, and complex sentence comprehension tests.

According to an embodiment, the comprehension score counts a number of correct responses in relation to word comprehension tests, a number of correct responses in relation to simple sentence comprehension tests, and a total number of correct responses for the cognitive test sequence.

According to an embodiment, the method comprises, following the execution of the test sequence, steps executed by the computer, comprising: if the comprehension score indicates that the total number of correct answers is greater than a first threshold value, selecting a first communication interface providing access to commands distributed over an unlimited number of pages; otherwise, if the comprehension score indicates that the number of correct answers in relation to the simple sentence comprehension tests is greater than a second threshold value, selecting a second communication interface providing access to commands distributed over several pages and reachable in no more than six selections using the control device; and otherwise, if the comprehension score indicates that the number of correct answers in relation to the simple word comprehension tests is greater than a third threshold value, selecting a third communication interface providing access to up to ten commands; otherwise waiting for a new test sequence to be executed.

According to an embodiment, the method comprises computer-executed steps of: selecting a sequence of cognitive tests to determine a patient's comprehension score, from a plurality of sequences of cognitive tests, or selecting a number of slides from groups of slides of a same difficulty level to form a sequence of cognitive tests to determine a patient comprehension score.

According to an embodiment, the selection of a sequence of cognitive tests is performed by excluding one or two most recent previously selected sequences of cognitive tests.

According to an embodiment, the method comprises computer-executed steps of: detecting by an eye-tracking device that the patient has maintained his gaze on a first area of interest in a page displayed on the screen for a duration greater than a temporal threshold value; and controlling the emission of a vocal message corresponding to the first area of interest, or displaying a new page related to the first area of interest.

According to an embodiment, the method comprises computer-executed steps of: detecting by an eye-tracking device a position in the display screen gazed at by the patient; displaying an icon at the detected position; determining that the detected position is static; when the detected position is static, animating the icon to indicate a time remaining to gaze at the static position; and activating a command corresponding to the detected static position when the elapsed time since the position is detected as static exceeds a time threshold value.

According to an embodiment, the time threshold value is set as a function of the selected communication interface, and/or as a function of the comprehension score obtained during the last performed cognitive test sequence.

According to an embodiment, the third communication interface provides access to a control module enabling direct control of external devices located in the patient's immediate environment.

Embodiments may also relate to a communication device comprising a computer, a display screen, and a control device, wherein the computer is configured to implement the method defined above.

According to an embodiment, the control device comprises an eye-tracking device supplying the computer with successive positions of the patient's gaze on the screen, the computer being configured to activate a command associated with an area of interest displayed on the screen, when the position of the patient's gaze supplied by the eye-tracking device is maintained in the area of interest for a duration greater than a time threshold value.

Non-limiting examples of the invention will be described in the following, in relation to the attached figures among which:

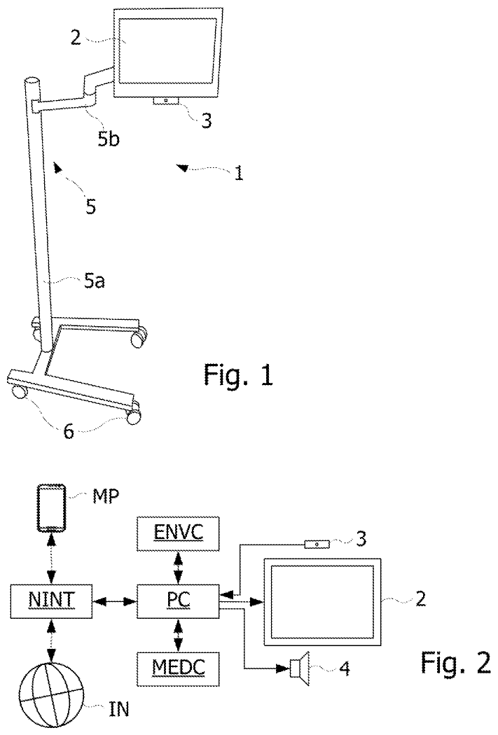

FIG. 1 schematically shows an embodiment of a communication aid device for bedbound, speech-impaired patients,

FIG. 2 schematically shows a hardware architecture of the communication aid device, according to an embodiment,

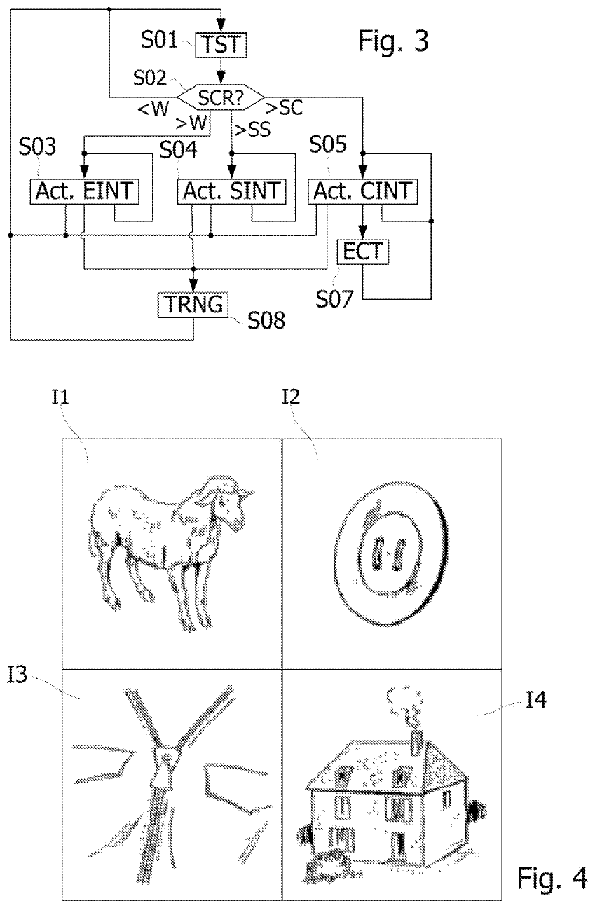

FIG. 3 schematically represents a functional architecture of the communication aid device, according to an embodiment,

FIGS. 4 to 6 show examples of pages displayed on a display during a cognitive test, according to an embodiment,

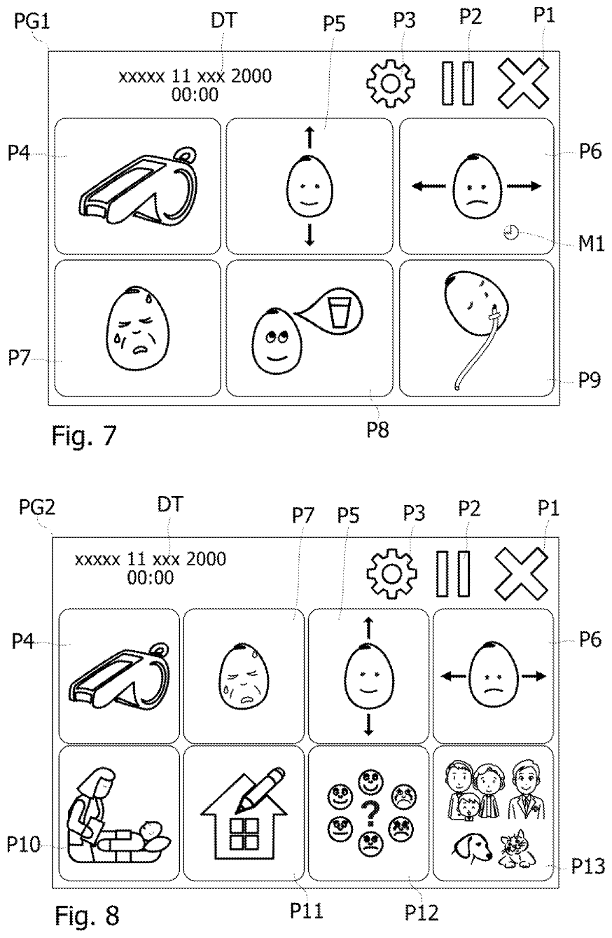

FIGS. 7 to 15 show examples of pages displayed by the communication aid device in an embodiment.

FIG. 1 shows a communication aid device 1. In an embodiment, the device 1 is suitable for bedbound, speech-impaired patients. FIG. 2 shows the hardware architecture of device 1. In FIGS. 1 and 2, device 1 comprises a PC connected to a display screen 2, a loudspeaker 4, a control device 3 such as an eye-tracking device, and a communication circuit NINT. The display screen 2 is attached to an adjustable support 5, configured to allow the display screen 2 to be positioned and oriented at a certain distance in front of the patient's face, regardless of whether the patient is lying down or sitting up. The support 5 may comprise a vertical axis 5a, an articulated arm 5b attached to the vertical axis so as to be adjustable in height, and a fastener (not shown) for securing the screen 2 to the end of the arm 5b. The fastener may comprise a ball-and-socket joint to facilitate orientation of the screen 2 towards the patient's face. Stand 5 may be mounted on castors 6. The PC may be a personal computer or a tablet, for example a touch-screen tablet.

The eye-tracking device 3 may be associated with the display 2 and comprise one or more image sensors (e.g. infrared or visible light) connected to an image analysis device configured to detect a user's eyes and determine a gaze direction, and possibly other phenomena such as eye blinking. The eye-tracking device 3 can also take the form of glasses to be worn by the patient, or any other device with the function of detecting the position of a point observed by a person. The image analysis device can also be configured to detect other phenomena that can be used to determine a patient's state of alertness or, more generally, communication capabilities.

The communication circuit NINT may include a network card, such as Ethernet, WiFi, Bluetooth, etc., to connect, for example, to the Internet network IN and/or a cell phone MP.

The PC can also be connected to medical devices MEDC and external devices ENVC. Medical devices may include equipment for measuring heart rate (electrocardiogram), respiratory rate, oxygen saturation or brain waves (electroencephalogram). External devices ENVC may include a TV set, a radio, a remote control for closing a window in the patient's room, a remote control for tilting the patient's bed, a remote control for air conditioning the room, a remote control for calling a caregiver. The computer PC can also be connected to an image sensor to receive images of the patient's face or of the patient as a whole and be configured to perform an analysis of the images of the patient's face in order to assess the pain felt, and/or to assess the patient's ability to use his or her hand to use a pencil or a computer keyboard.

Other control devices can be connected to the PC, such as a mouse, keyboard or touch screen, to enable a caregiver to operate the computer. A push-button can also be connected to the PC to enable the patient to validate a selection or trigger an alarm, if he or she is able to do so.

FIG. 3 shows an example of the functional architecture of a program executed by the computer PC, according to an embodiment. The program includes a test module TST, several communication interface modules EINT, SINT, CINT, an external equipment control interface module ECT, and a cognitive training module TRNG.

The communication interface modules may include an elementary interface module EINT, a simple interface module SINT, and a complex interface module CINT.

When the program is initiated, the PC runs the test module TST in step S01. The test module TST runs a sequence of tests of the patient's cognitive and communication skills, and calculates a score SCR based on the answers provided by the patient. In an embodiment, the sequence of cognitive tests is adapted to the patient's condition (critical care) and to the use of an eye-tracking device. The tests carried out by the test module TST are configured so that they can be performed regardless of the patient's position (lying down, sitting up), and on several occasions during the patient's hospitalization. The duration of the tests is therefore chosen to be sufficiently short, for example less than 5 minutes. At the end of a test session, the test module can display and/or vocally announce the test result, archive it and send it to a remote computer.

At any time, a caregiver can assess the patient's physical capabilities to determine the most suitable control device. For example, if the patient does not have the use of the fingers of one hand, the eye-tracking device alone would appear to be the most suitable. If the patient can press a push-button, this device can be combined with the eye-tracking device to validate an observed area of interest. If the patient can move one arm and one hand, he or she can use an independent keyboard or touchpad, or a touchscreen placed on the display screen 2.

In step S02, the score SCR is compared with threshold values to activate a communication interface selected from several predefined communication interfaces according to the score. So, for example, if the score SCR is below a threshold value W, the PC does not activate any communication interface. If the score SCR is higher than the value Wand lower than a threshold value SS, the PC activates the elementary interface module EINT, in step S03. If the score SCR is greater than the threshold value SS, but less than a value SC, the PC activates the simple interface module SINT, in step S04. If the score SCR is higher than the SC value, the PC activates the complex interface module CINT, in step S05. In this way, the patient has access to a communication interface whose complexity is selected on the basis of an assessment of his/her cognitive and communication abilities.

Each of the interface modules EINT, SINT, CINT is configured to present command icons associated with a command and possibly with textual information indicating the meaning of the icon, and to consider and display the patient's eye path on screen 2, i.e. the successive positions of a point on screen 2 observed by the patient.

In an embodiment, the position of the point viewed by the patient on screen 2 is indicated by a pointer (M1 in FIG. 7). When this point is static, the pointer M1 may be animated to indicate the time remaining for the patient to maintain his or her gaze in order to select a displayed icon and thus activate the corresponding command. The time required to gaze at an icon to activate the corresponding command may be set according to the patient's condition, for example between 1 and 2.5 s, or may depend on the communication interface activated. According to an example, the icon gaze time may be maximum (e.g. 2.5s) when the communication interface EINT is selected, minimum (e.g. 1s) when the communication interface CINT is selected, and an intermediate value, e.g. between 1.5 and 2 s, when the communication interface SINT is selected.

The EINT, SINT and CINT interface modules differ from one another in terms of the commands to which they provide access, the number of command icons displayed simultaneously, and the maximum number of selections to be made to access a command.

The EINT, SINT and CINT interface modules display a home page PG1, PG2 and PG10 respectively (FIGS. 7, 8 and 14), with command icons providing access to various commands enabling the patient to communicate with people around him/her. Each command icon may be located in an area of interest, where the patient's gaze is maintained to activate the corresponding command. The icons presented may be animated, in particular to improve their intelligibility, to determine the command assigned to them.

Each home page PG1, PG2, PG10 displayed by the EINT, SINT and CINT interface modules may indicate the date, time and location, allowing the patient to situate him/herself in time and space.

Among the control icons presented on the respective home pages of the interface modules EINT, SINT and CINT, an icon may be provided to activate the cognitive training module TRNG, at step S08. The module TRNG may also be activated at the end of a cognitive test sequence executed by the test module TST. At the end of a training phase performed by the module TRNG, or from the home screen pages displayed by the EINT, SINT and CINT interface modules, the patient may also activate the test module TST to perform a new cognitive test (activate the test module TST).

The cognitive training module TRNG may offer the patient a rehabilitation or re-education program, which can be defined according to the last score SCR obtained at the end of a test sequence run by the test module TST. The module TRNG may use virtual reality. In this way, during each session of the rehabilitation program, the module TRNG can place the patient in an environment that enables him or her to perform simple acts of daily life and/or acts of increasing complexity, particularly in order to reduce disturbances to the sleep/wake cycle. The acts of daily life are, for example, fetching bread, going to the hairdresser, taking children to school. The rehabilitation program is adapted to the patient's characteristics (age, gender, socio-professional status, family environment, etc.), the score obtained in the last test sequence run by the test module TST and the progression of these results. The duration of each session of the rehabilitation program is adapted to the patient's attention span and state of weakness. This duration can be determined at the patient's discretion or according to the last score SCR determined by the test module TST.

The interface module CINT provides access to a control module ECT (step S07) for direct control of external devices located in the patient's immediate environment, for example via an infrared or radio link. These external devices may include, for example, a TV set, a radio set, a room temperature control device, a remote control for window shutters, a remote control for the ambient light intensity, a remote control for tilting a bed or chair, and other interactions with the environment, the care team, and biomedical devices such as a morphine pump.

In an embodiment, a calibration module may be activated when the communication device is initialized before the test module TST is activated. The calibration module is configured to control the brightness of the image of the patient's face and adjust the distance between the patient's face and the display screen 2. The calibration step can be followed by the selection of a control device adapted to the patient's condition. This control device may be the eye-tracking device 3 alone or in combination with a push-button in the patient's hand, to be used to validate a selection made using device 3. If the patient has full use of an arm and a hand, the control may be a keyboard, a touchpad or a touchscreen placed on display screen 2.

In an embodiment, the test module TST is configured to successively display on screen 2 slides from a series of slides divided into areas of interest, for example from 2 to 4 areas of interest. When a slide is displayed, a voice command designating one of the areas of interest is broadcast by loudspeaker 4. The vocal instruction may be a recording of a real human voice. The patient must place his or her gaze on the area of interest designated by the vocal instruction. When a slide display time has elapsed, a new slide is displayed, and a new voice command is issued. As the slides are displayed, the test module TST captures the patient's responses and calculates a score SCR incrementally according to these responses.

The series of test slides, each associated with a set of instructions, is designed to test the patient's cognitive abilities, such as oral comprehension and communication skills, and the patient's ability to fix his or her gaze on a limited area of screen 2.

In an embodiment, each slide in the series of slides displayed in succession is associated with a level of difficulty. The slides in the series can be ordered by increasing level of difficulty or presented in random order. The order in which the slides are presented can also be defined on the fly, based on the answers already provided by the patient during the test. For example, the series of slides has three levels of difficulty to assess the patient's listening comprehension ability, namely a first level of word comprehension, a second level of comprehension of simple sentences, and a third level of comprehension of more complex sentences.

FIG. 4 shows an example of a slide at the first difficulty level, displayed by the test module TST during step S01. This slide comprises four areas of interest I1-I4, each presenting an image, namely the image of a sheep I1, a button 12, a zipper 13 and a house 14. The vocal instruction broadcast when the slide is displayed is, for example, “look as long as possible at the button” or simply “button”.

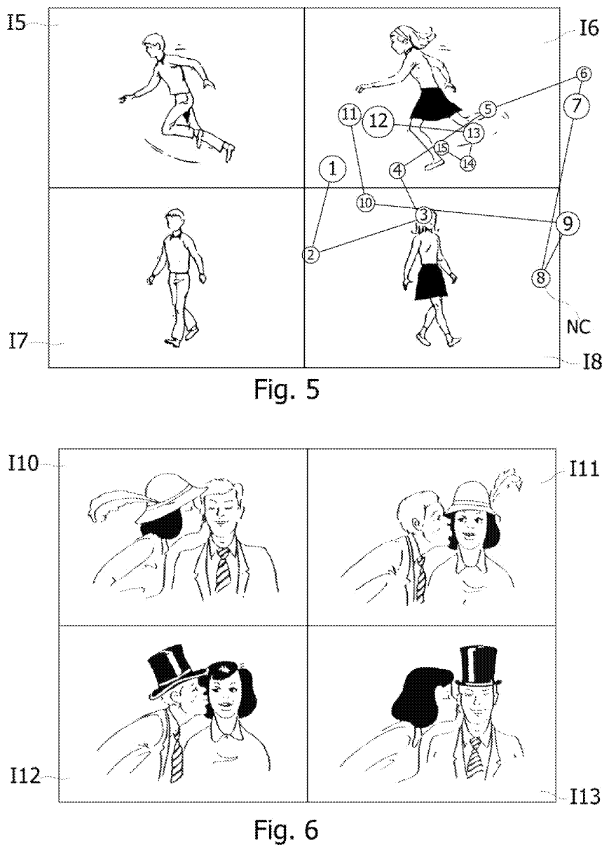

According to an embodiment, the vocal instructions of the second difficulty level comprise sentences with only a subject and a verb, such as “the girl walks”, “the man eats”, “the woman drinks”. Voice instructions at the second level of difficulty may also include sentences with a subject, an active verb and an object, such as “the boy follows the dog”, “the horse pulls the boy”, “the woman follows the dog and the car”. FIG. 5 shows an example of a slide at the second level of difficulty. This slide contains four images 15-I8, i.e. a boy running 15, a girl running 16, a boy walking 17 and a girl walking I8. The vocal instruction broadcast when the slide is displayed is, for example, “look as long as possible at the girl running”.

In an embodiment, voice instructions for the third level of difficulty include sentences with an object location, such as “the cat is behind the chair”, a passive verb, such as “the dog is being pushed by the boy”, and sentences with a subordinate clause, such as “it's the boy who is looking at the dog”, “the man wearing the hat is kissing the woman”. FIG. 6 shows an example of a slide at the third level of difficulty. This slide comprises four images I10-I13, namely image I10 of a woman wearing a hat and kissing a man, image I11 of a man kissing a woman wearing a hat, image I12 of a man wearing a hat and kissing a woman, and image I13 of a woman kissing a man wearing a hat. The vocal instruction broadcast when the slide is displayed is, for example, “look as long as possible at the man wearing a hat kissing the woman”. According to an embodiment, the test module TST is configured to display each slide for a certain duration, for example 6 seconds, and record the patient's eye paths in relation to each displayed slide.

In an embodiment, the computer PC is configured to analyze the recorded eye paths to determine the score SCR. The eye path recording may include colored discs, numbered in chronological order and whose size corresponds to the time the patient's gaze was maintained on the center of the disc. The color of each disc indicates whether the disc is on the image corresponding to the correct response or not. For example, disks on the image corresponding to the correct answer are green, and disks on other images are red. In the example shown in FIG. 5, the eye path comprises 15 positions NC numbered from 1 to 15. Positions 1, 4, 5 and 11 to 15 are in the area of interest corresponding to the correct answer.

The eye-tracking analysis may include a calculation of the time during which the patient has maintained his/her gaze on the area of interest corresponding to the instruction. In this way, the patient can be considered to have given a good response if he/she has maintained his/her gaze on the area of interest corresponding to the vocal instruction for a time greater than a threshold value. This threshold value is set, for example, at half the slide display time.

The test module TST may also be configured to distinguish between wrong and no responses. A wrong response occurs when the patient has selected or maintained his/her gaze on an area of interest that does not correspond to the vocal instruction for a time greater than the threshold value. An absence of response occurs when the patient has not selected an area of interest in the allotted time, or has looked at the displayed slide for less than the threshold value.

The test module TST is configured to count correct answers according to the level of difficulty associated with each slide. The test module TST may also be configured to count wrong answers according to the level of difficulty associated with each slide. The test module TST may also be configured to count the absences of answers.

In an embodiment, the test module TST is configured to select a series of slides to be displayed successively from a number of alternative series memorized by the computer PC, to enable the patient to perform the test several times during his or her stay while avoiding a learning phenomenon. The series of slides to be displayed may be selected randomly from the series of slides available, excluding the last one or two series of slides previously selected.

In an embodiment, each of the slides in the slide series is associated with several voice prompts, with one of the associated voice prompts being randomly selected when the slide is displayed.

In another embodiment, the test module TST is configured to dynamically generate the series of slides to be displayed successively, by randomly selecting a number of slides from groups of slides of the same difficulty level. Slides selected during the last one or two tests performed may be excluded from the selection.

At the end of a test, the score SCR may reveal four possible situations:
Case 1, the patient understands complex sentences, for example when he/she has provided fewer than three incorrect answers for all the slides in the test sequence,
Case 2, the patient understands simple sentences, for example when he/she has provided fewer than two incorrect answers for the second level of simple sentence comprehension,
Case 3, the patient understands words, for example when he or she has provided a correct answer for each of the slides in the first word comprehension level,
Case 4, the patient is unable to use the communication device, e.g. when he/she has provided at least one incorrect answer for the first-level slides or has not looked at the slides.

In case 1, the complex interface module is activated CINT. In case 2, the simple interface module SINT is activated. In case 3, the elementary interface module EINT is activated. In case 4, none of the interface modules EINT, SINT, CINT is activated, as the patient is considered unable to use the communication device.

According to an embodiment, the elementary interface module EINT is configured to display a single command page providing access to a limited number of commands allowing the patient to communicate about elementary needs.

According to an embodiment, the intermediate interface module SINT is configured to display a first control page providing access to a larger number of commands that can provide access to up to three further successive command pages, so that all accessible commands can be activated in up to six selections from the home page.

In an embodiment, the complex interface module CINT complements the commands accessible via the simple interface module SINT by providing access to a keyboard, the Internet and the patient's telephone. The complex interface module CINT may also provide direct access to the control module ECT.

In an embodiment, the computer PC is configured to analyze the recorded eye paths and determine a gaze time for a displayed icon to trigger the command associated with the icon.

FIGS. 7 to 15 show various pages PG1-PG8, PG10, PG11 displayed by one or other of the communication interface modules. These pages generally comprise a top banner and areas of interest each featuring a pictogram or icon, which may be associated with textual information indicating the command associated with the pictogram.

FIG. 7 shows an example of the home page PG1 displayed by the elementary interface module SINT. The home page PG1 comprises a top banner and six areas of interest P4, P5, P6, P7, P8. The top banner displays the current date and time DT, and includes three pictograms P1, P2, P3 associated respectively with commands to exit from the communication application P1, pause the eye-tracking device 3 P2, and access other functionalities of the communication device P3. These other functionalities may include activating the test and cognitive training modules TST, TRNG, activating a calibration module for the eye-tracking device 3, and defining operating parameters for the communication device. The area of interest P4 is associated with an alarm trigger to enable the patient to request urgent intervention by a caregiver. This alarm may be emitted in audible form and/or transmitted to the room's existing alarm system and/or transmitted to a remote computer.

Areas of interest P5 and P6 enable the patient to answer “YES” and “NO”, respectively, to a question asked orally by a person standing next to him or her. Area of interest P7 enables the patient to signal that he or she is experiencing pain. Area of interest P8 allows the patient to signal thirst. Area of interest P9 allows the patient to signal that the intubation tube is bothering him/her. Following the selection of one of the areas of interest P4-P9, the communication device may control a spoken broadcast of the text corresponding to the area of interest.

FIG. 8 shows an example of a home page PG2 displayed by the simple interface module SINT. The home page PG2 includes a top banner and eight areas of interest, including areas of interest P4, P7, P5 and P6 from FIG. 7, and areas of interest P10, P11, P12 and P13. Each area of interest P10-P13 also features a pictogram which may be associated with textual information indicating the command associated with the pictogram. The top banner may be identical to that of page PG1.

Each of the areas of interest P7 and P10 to P13 provides access to a command page containing selectable common questions asked by critical care patients. The area of interest P7 provides access to a communication page enabling patients to specify how they perceive pain. Area of interest P10 provides access to a communication page enabling the patient to request special care, such as cleansing, massage, scent, or music. The area of interest P11 provides access to a communication page enabling the patient to request an action on his or her immediate environment, such as the bed, light, noise, or ambient temperature. Area of interest P12 provides access to a communication page enabling the patient to report discomfort, for example relating to thirst, hunger, heat, sleep, breathing, mood, or relating to the intubation tube. The area of interest P13 provides access to a communication page enabling the patient to select a relative or caregiver with whom he or she wishes to communicate.

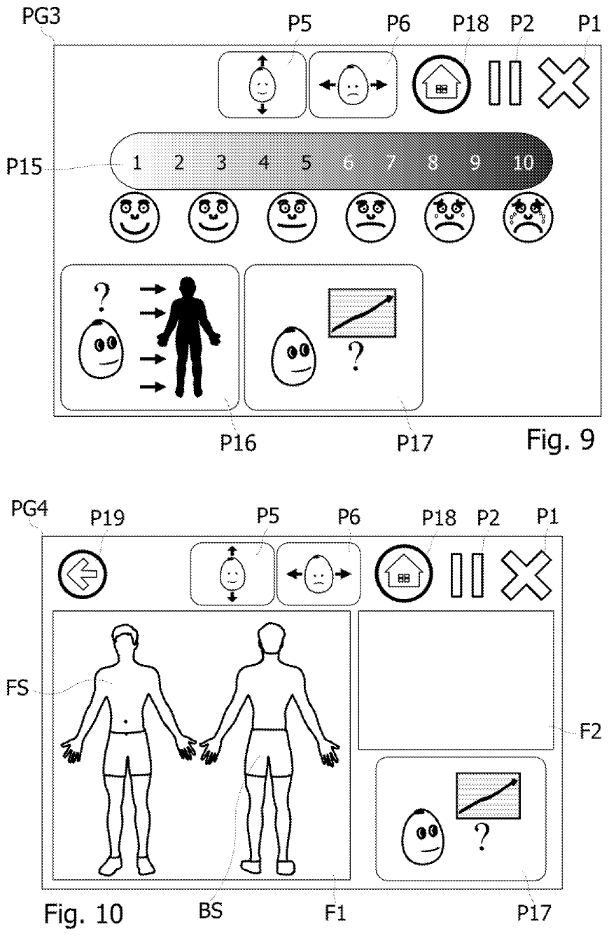

FIG. 9 shows an example of a communication page PG3 about pain, accessible from the area of interest P7 (FIG. 8). The page PG3 includes a banner and three areas of interest P15, P16 and P17. The banner includes the control icons P1, P2 described above, the areas of interest P5, P6 described above, for yes/no answers, and an icon P18 for displaying the home page PG2. The area of interest P15 represents a scale graduated from 1 to 10, in which the patient can designate a point to express a degree of pain felt between 1 and 10. The selection of such a point triggers an oral broadcast of the value of the designated point. The area of interest P16 provides access to a communication page enabling the patient to indicate the location of the painful region. Area of interest P17 provides access to a page enabling the patient to communicate how he/she perceives this pain.

FIG. 10 shows an example of a communication page PG4 about the location of the painful region, this page being displayed following the designation of the area of interest P16 (FIG. 9). Page PG4 includes a banner, a window F1 showing the front FS and rear BS of a human body, a text display window F2 and the area of interest P17. The banner includes that of page PG3 and an icon P19 for returning to the previous page PG3. The front FS and rear BS panels allow the patient to designate the painful area of his/her body. Following selection of a body region, this region may be signaled in the form of spoken text and/or displayed in the window F2. The front and back panels FS, BS presented may be adapted to the patient's gender and/or age.

FIG. 11 shows an example of a communication page PG5 about how pain is experienced, displayed following the designation of the area of interest P17 in page PG3 or PG4. The page PG5 includes a banner, the area of interest P16 from page PG3, and eleven areas of interest P20 to P29. The banner is identical to the banner of page PG4. Areas of interest P20 and P21 indicate that pain is felt continuously or discontinuously, respectively. Areas of interest P22, P23 indicate that the pain appears abruptly or progressively, respectively. Areas of interest P24, P25, P26, P27, P28 indicate sensations of itching, burning, stinging, numbness, or cramping, respectively. Area of interest P29 is used to indicate that the designated region is insensitive.

In this way, the patient can precisely describe the pain experienced by performing six selections of areas of interest on pages PG2, PG3, PG4, PG5.

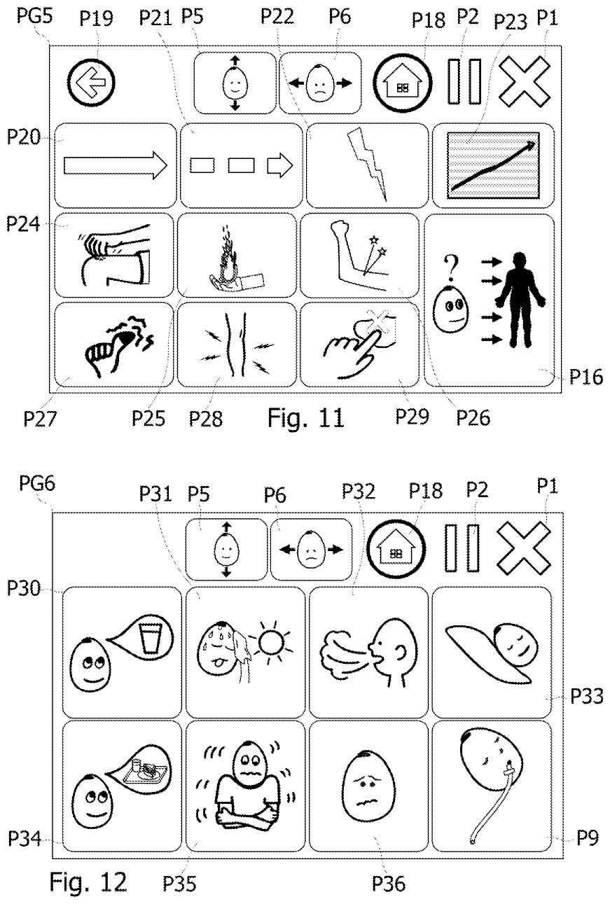

FIG. 12 shows an example of a communication page PG6 allowing the patient to indicate a sensation, this page being displayed following the designation of the area of interest P12 on the page PG2. The page PG6 comprises a banner and eight areas of interest P9 and P30 to P36, including the area of interest P9 described above (FIG. 7). The banner is identical to that of page PG3. Areas of interest P30, P34 allow the patient to indicate thirst and hunger, respectively. Areas of interest P31, P35 allow the patient to indicate that he/she is too hot or cold, respectively. Areas of interest P32, P33 allow the patient to indicate that he/she is breathless or sleepy, respectively. Area of interest P36 allows the patient to indicate anxiety or depression.

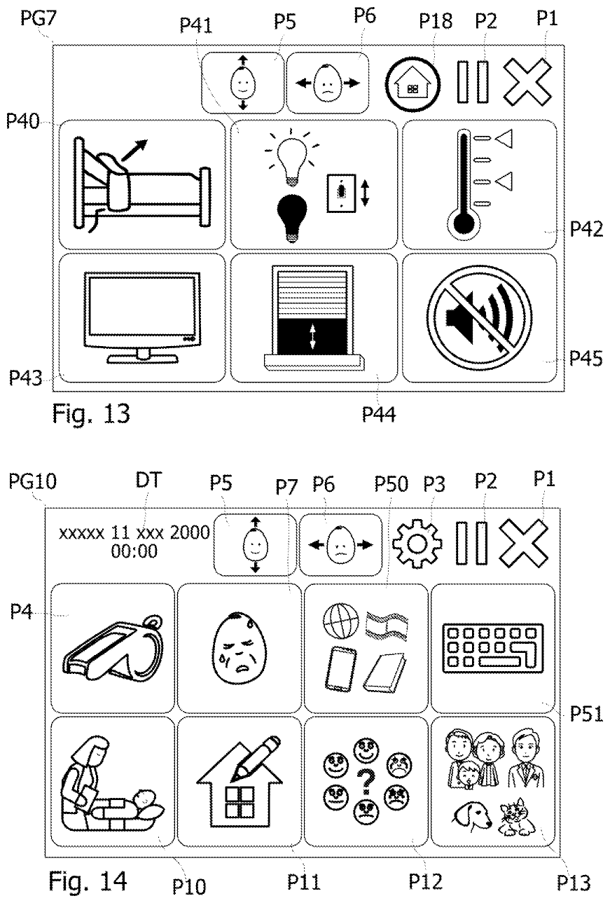

FIG. 13 shows an example of a communication page PG7 allowing the patient to express a need concerning his/her environment. The page PG7 is displayed following the designation of the area of interest P11 on the page PG2. This page includes a banner and six areas of interest P40 to P45. The banner is identical to that of page PG3. Area of interest P40 allows the patient to indicate that he/she wishes to change position on the bed or change the bed's inclination. Areas of interest P41 and P44 are used to request that the light be switched on or off, and that the window shutter be opened or closed, respectively. Area of interest P42 allows the patient to indicate that he/she wishes to change the temperature setting of the room's heating or air-conditioning system. Area of interest P43 allows the patient to indicate that he/she wishes to watch television. Area of interest P45 allows the patient to indicate that he/she is bothered by ambient noise.

FIG. 14 shows an example of the home page PG10 displayed by the complex interface module CINT. The home page PG10 comprises a top banner and eight areas of interest including areas of interest P4, P7, P10, P11, P12 and P13 from the home page PG2, and areas of interest P50, P51. The top banner displays the current date and time DT, the three pictograms P1, P2, P3, and the areas of interest P5, P6 allowing the patient to answer “YES” and “NO”, respectively, to a question asked vocally. Areas of interest P4, P7, P10, P12 and P13 provide access to the same pages as those of the home page PG2 of the simple interface module SINT. Area of interest P50 provides access to a control page for selecting external communication systems, such as the Internet IN, the patient's cell phone MP, and a library of multimedia documents, such as books or films. The connection to the patient's phone may provide access to the phone's functionalities. Area of interest P51 provides access to a keyboard. The area of interest P11 displays the page PG7 for selecting an external device to be controlled, presented in the areas of interest P40 to P44 (bed, lighting, shutters, air conditioning, etc.). Selecting one of these external devices from the page PG7 activates the module ECT for direct control of these devices.

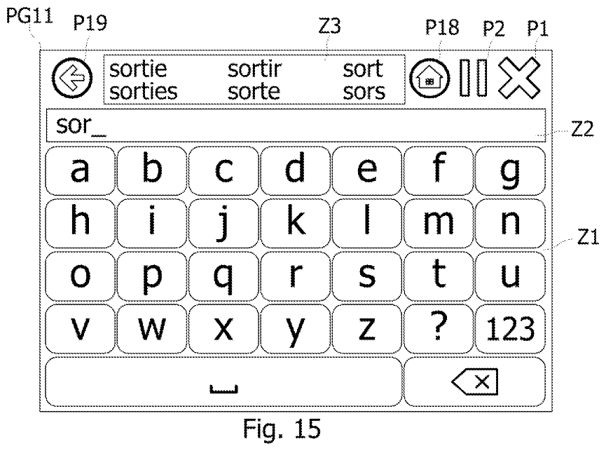

The banner of each page displayed from the home page PG10 can include the area of interest P51, providing access to the keyboard (FIG. 15).

FIG. 15 shows an example of a page PG11 featuring a keyboard Z1, which is displayed following the selection of the area of interest or icon P51. The page PG11 also includes a banner featuring the control icons P1, P2, P18 and P19 already described, as well as display zones Z2, Z3. Display zone Z2 shows characters selected using the keyboard, or words selected in display zone Z3. Display zone Z3 shows word suggestions, selected by the communication device according to the characters displayed in display zone Z2 for the word currently being entered. By designating the zone Z2, the word or phrase displayed in this zone may be broadcast vocally.

The home page displayed by the complex interface module CINT thus provides access to extensive communication resources.

In an embodiment, the different communication interfaces may be presented in different ways, for example with a distinct background color, so that on each displayed page one can see which communication interface the page belongs to.

It will be clear to those skilled in the art that the present invention may be subject to various alternatives and applications. In particular, the invention is not limited to the provision of three communication interface modules or three communication interfaces. In fact, two or more separate communication interfaces may be provided to suit patients' abilities to use such interfaces.

The cognitive tests described above are only examples, and other forms of testing may be organized to determine the patient's ability to understand instructions provided orally and on the screen displays, and to designate areas of interest on a computer screen.

The control device used by the patient to designate areas of interest is not necessarily an eye-tracking device, and the patient to whom the communication device is addressed is not necessarily unable to move a hand. The control device may therefore be any device that allows the patient to designate an area of a page displayed on a screen.

In some situations, the support 5 may be omitted, particularly when the patient can hold a tablet in his/her hands.

1. A communication method, comprising the following steps carried out by a computer:
providing a patient with a control device and a display screen connected to the computer,
executing a sequence of cognitive tests during which the patient interacts with the computer using the display screen and the control device, to determine a comprehension score (SCR) for the patient,
selecting a communication interface as a function of the comprehension score, from a plurality of communication interfaces (EINT, SINT, CINT) implemented by the computer and having distinct respective complexities, each communication interface including at least one home page (PG1, PG2, PG10) displayable on the display screen and including selectable areas of interest (P1-P13, P50, P51) using the control device,
activating the selected communication interface and displaying the home page of the selected communication interface,
detecting a designation by the control device of an area of interest among the areas of interest in a page (PG1-PG7, PG10, PG11) displayed on the display screen, and
controlling the emission of a voice message following detection of the designation of an area of interest.

2. The method according to claim 1, wherein the communication interfaces (EINT, SINT, CINT) comprise:
a first communication interface (EINT) comprising only a home page (PG1) providing access to at most ten commands, and/or
a second communication interface (SINT) providing access to commands from areas of interest distributed over the home page (PG2) and several other pages (PG3-PG7), the commands of the second communication interface being available in at most six selections using the control device, and/or
a third communication interface (CINT) providing access to commands distributed over an unlimited number of pages.

3. The method according to claim 1, wherein each of the cognitive tests comprises steps of:
displaying a page comprising several images (I1-I8, I10-I13) on the display screen,
issuing a voice command in relation to the images on the displayed page,
acquiring a response from the patient in relation to the page displayed, using the control device, and
updating the comprehension score (SCR) as a function of the patient's response, wherein the patient's response is a selection signal for an area of the display screen or eye movements detected using an eye-tracking device.

4. The method according to claim 3, comprising the incrementation by the computer of the score (SCR) when the patient has maintained his/her gaze on one of the images (I1-I8, I10-I13) displayed, corresponding to the vocal instruction.

5. The method according to claim 1, wherein the cognitive tests comprise word comprehension tests, simple sentence comprehension tests, and complex sentence comprehension tests.

6. The method according to claim 5, wherein the comprehension score (SCR) counts a number of correct responses in relation to word comprehension tests, a number of correct responses in relation to simple sentence comprehension tests, and a total number of correct responses for the cognitive test sequence.

7. The method according to claim 6, comprising, following the execution of the test sequence, steps executed by the computer, comprising:
if the comprehension score (SCR) indicates that the total number of correct answers is greater than a first threshold value, selecting a first communication interface (CINT) providing access to commands distributed over an unlimited number of pages,
otherwise, if the comprehension score indicates that the number of correct answers in relation to the simple sentence comprehension tests is greater than a second threshold value, selecting a second communication interface (SINT) providing access to commands distributed over several pages and reachable in no more than six selections using the control device, and
otherwise, if the comprehension score indicates that the number of correct answers in relation to the simple word comprehension tests is greater than a third threshold value, selecting a third communication interface (EINT) providing access to up to ten commands,
otherwise waiting for a new test sequence to be executed.

8. The method according to claim 1, comprising computer-executed steps of:
selecting a sequence of cognitive tests to determine a patient's comprehension score, from a plurality of sequences of cognitive tests, or
selecting a number of slides from groups of slides of a same difficulty level to form a sequence of cognitive tests to determine a patient comprehension score.

9. The method according to claim 8, wherein the selection of a sequence of cognitive tests is performed by excluding one or two most recent previously selected sequences of cognitive tests.

10. The method according to claim 1, comprising computer-executed steps of:
detecting by an eye-tracking device that the patient has maintained his/her gaze on a first area of interest (P1-P13) in a page (PG1-PG7, PG10, PG11) displayed on the screen for a duration greater than a temporal threshold value, and
controlling the emission of a vocal message corresponding to the first area of interest, or displaying a new page related to the first area of interest.

11. The method according to claim 1, comprising computer-executed steps of:
detecting by an eye-tracking device a position in the display screen gazed at by the patient,
displaying an icon (M1) at the detected position,
determining that the detected position is static,
when the detected position is static, animating the icon to indicate a time remaining to gaze at the static position, and
activating a command corresponding to the detected static position when the elapsed time since the position is detected as static exceeds a time threshold value.

12. The method according to claim 11, wherein the time threshold value is set as a function of the selected communication interface (EINT, SINT, CINT), and/or as a function of the comprehension score (SCR) obtained during the last performed cognitive test sequence.

13. The method according to claim 1, wherein the third communication interface (CINT) provides access to a control module (ECT) enabling direct control of external devices (ENVC, MEDC) located in the patient's immediate environment.

14. A communication device comprising a computer (PC), a display screen (2), and a control device (3), wherein the computer is configured to implement the method according to claim 1.

15. The device according to claim 14, wherein the control device comprises an eye-tracking device supplying the computer (PC) with successive positions of the patient's gaze on the screen, the computer being configured to activate a command associated with an area of interest (P1-P13) displayed on the screen, when the position of the patient's gaze supplied by the eye-tracking device is maintained in the area of interest for a duration greater than a time threshold value.
